(19) United States
(12) Patent Application Publication
(10) Pub. No.: US 2023/0389873 A1
(43) Pub. Date: Dec. 7, 2023

(54) Infrared Glucose Monitoring Wristband Device

(71) Applicant: Martin Johnson, Lynden, WA (US)

(72) Inventor: Martin Johnson, Lynden, WA (US)

(21) Appl. No.: 17/829,686

(22) Filed: Jun. 1, 2022

(51) Int. Cl. A61B 5/00; A61B 5/145; A61B 5/1455

(52) U.S. Cl. CPC A61B 5/681; A61B 5/14532; A61B 5/1455; A61B 5/0002; A61B 5/748

(57) ABSTRACT

An infrared glucose monitoring wristband device for measuring blood glucose levels includes a wristband having a smart watch. A first side of the wristband has a plurality of holes and a second side of the wristband has a aperture. Each of the holes is a first portion of a wristband fastener. A glucose reader is a second portion of the wristband fastener and includes an encasing. The encasing has a top end, a bottom end, and an interior. The interior defines a space for a pair of infrared diodes to be positioned within. A pair of glass caps is positioned on the top end of the encasing, wherein the pair of glass caps emits infrared light upon the wrist of the user. A circuit board is positioned on the bottom end of the encasing and sends readings from the pair of infrared diodes to the smart watch.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to health monitoring wristband device and more particularly pertains to a new health monitoring wristband device for measuring blood glucose levels.

### (2) Description of Related Art Including Information Disclosed Under 37 CFR 1.97 and 1.98

The prior art relates to health monitoring wristband devices. The prior art relates to a variety of health monitoring wristband devices configured for reading blood glucose levels of the user. Known prior art lacks a health monitoring wristband device configured for reading blood glucose levels using infrared light emitting diodes.

### (h) BRIEF SUMMARY OF THE INVENTION

An embodiment of the disclosure meets the needs presented above by generally comprising a wristband including a smart watch. The wristband is configured for enwrapping the wrist of the user. A first side of the wristband has a plurality of holes and a second side of the wristband has a aperture. Each of the holes is a first portion of a wristband fastener. A glucose reader is a second portion of the wristband fastener. The glucose reader further includes an encasing. The encasing has a top end and a bottom end. Furthermore, the encasing is cylindrical and has an interior. The interior defines a space for a pair of infrared diodes to be positioned within. Each of the infrared diodes is configured to measure glucose levels within the wrist of the user. A pair of glass caps is positioned on the top end of the encasing, wherein the pair of glass caps is configured for emitting infrared light upon the wrist of the user. A circuit board is positioned on the bottom end of the encasing and is in electric communication with the pair of infrared diodes. The circuit board is configured for sending readings from the pair of infrared diodes to the smart watch.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of an infrared glucose monitoring wristband device according to an embodiment of the disclosure.

FIG. 2 is an isometric view of an embodiment of the disclosure.

FIG. 3 is a bottom isometric in-use view of an embodiment of the disclosure.

FIG. 4 is a cross-sectional view of an embodiment of the disclosure taken from FIG. 3 of Line 4-4.

FIG. 5 is a cross-sectional view of an embodiment of the disclosure taken from FIG. 4 of Circle 5.

### (j) DETAILED DESCRIPTION OF THE INVENTION

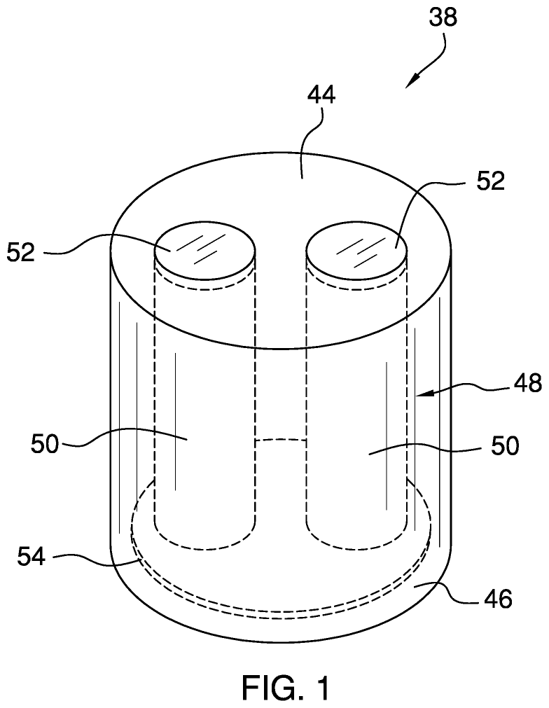

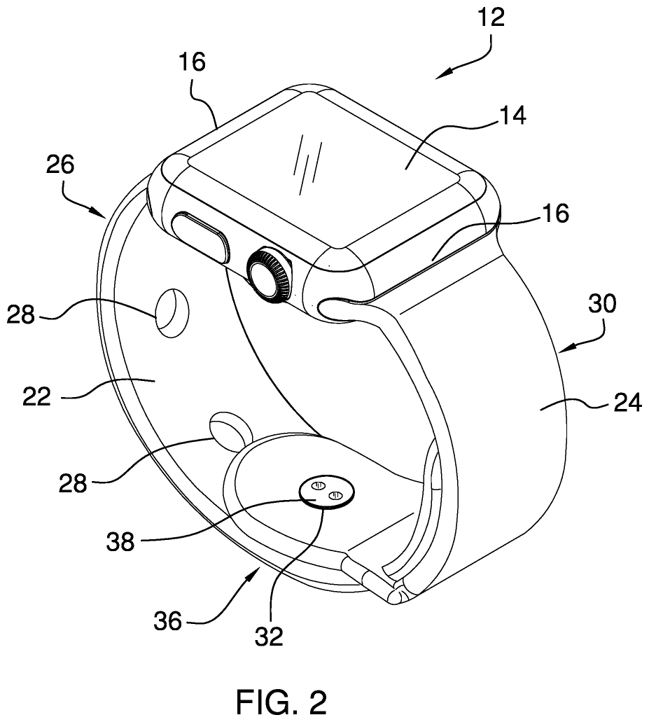

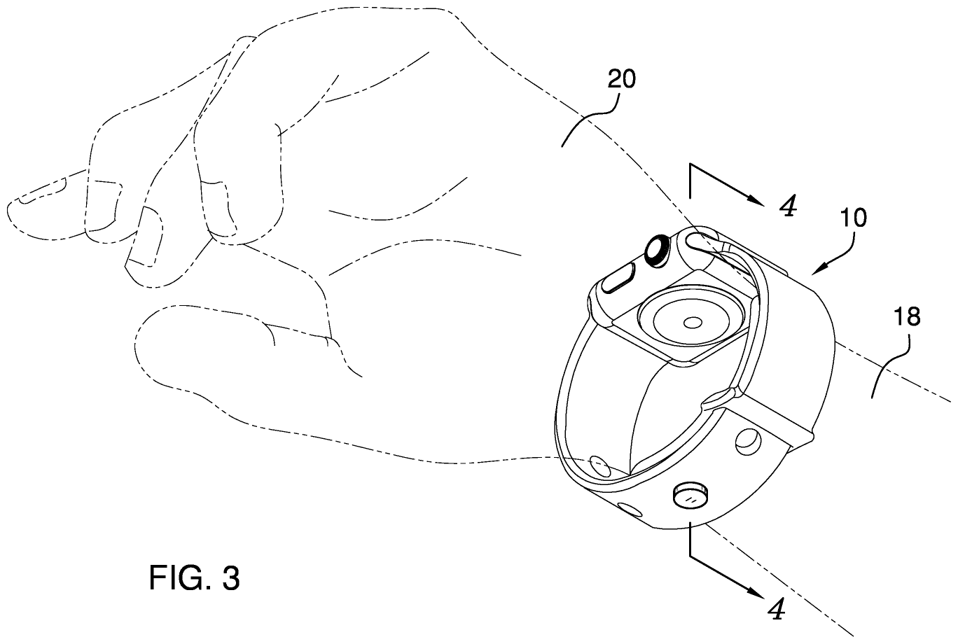

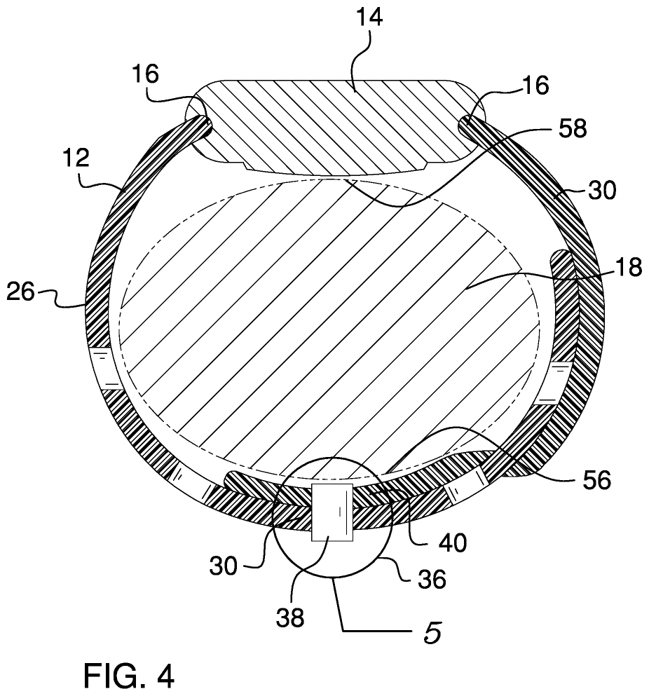

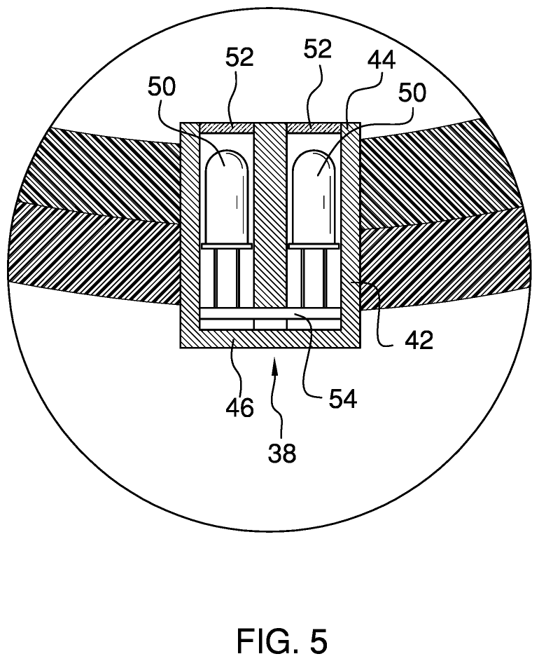

With reference now to the drawings, and in particular to FIGS. 1 through 5 thereof, a new health monitoring wristband device embodying the principles and concepts of an embodiment of the disclosure and generally designated by the reference numeral 10 will be described.

As best illustrated in FIGS. 1 through 5, the infrared glucose monitoring wristband device 10 generally comprises a wristband 12. The wristband 12 comprises a smart watch 14 configured for being a mobile device with touch screen capabilities. The smart watch 14 has a pair of sides 16, wherein the wristband 12 protrudes out from each of said sides 16 of said smart watch 14. The wristband 12 is configured for enwrapping the wrist 18 of the user 20. Furthermore, the wristband 12 has a wrist surface 22 and a watch surface 24, wherein the wrist surface 22 of the wristband 12 is configured for abutting a surface of the wrist 18 of the user 20.

A first side 26 of the wristband 12 has a plurality of holes 28 positioned in an array along the wristband 12. A second side 30 of the wristband 12 has a aperture 32 positioned at an end of the second side 30 of the wristband 12. The watch surface 24 of the second side 30 of the wristband 12 is positioned abutting the wrist surface 22 of the first side 26 of the wristband 12 when in-use. Each of the holes 28 is a first portion 34 of a wristband fastener 36, wherein the aperture 32 of the second side 30 becomes concentric to an in-use hole of the plurality of holes 28 of the first side 26.

A glucose reader 38 is a second portion 40 of the wristband fastener 36 and is configured for being positioned within the aperture 32 of the second side 30 and the in-use hole of the plurality of holes 28 of the first side 26 of the wristband 12 wherein retaining the wristband 12 in a fixed position around the wrist 18 of the user 20. The glucose reader 38 further includes an encasing 42 having a top end 44 and a bottom end 46. The encasing 42 is cylindrical and has an interior 48 defining a space for a pair of infrared diodes 50 to be positioned within.

Each of the infrared diodes 50 is configured to measure glucose levels within the wrist 18 of the user 20. The pair of infrared diodes 50 emits infrared light into the vein of the wrist 18 to determine the glucose level within the blood of the vein. Additionally, a pair of glass caps 52 is positioned on the top end 44 of the encasing 42. Each of the glass caps 52 is coupled to a respective on of each of the infrared diodes 50. Each of the glass caps 52 is configured for emitting infrared light upon the wrist 18 of the user 20. A circuit board 54 is positioned on the bottom end 46 of the encasing 42. The circuit board 54 is a transceiver and is in electric communication with the pair of infrared diodes 50. The circuit board 54 obtains the data of the glucose levels from the pair of diodes 50. Furthermore, the circuit board 54 is in wireless communication to the smart watch 14, wherein the circuit board 54 is configured for sending readings from the pair of infrared diodes 50 to the smart watch 14.

In use, the user 20 enwraps the wristband 12 around the wrist 18, wherein the glucose reader 38 is positioned on the palm side 56 of the wrist 18 and the smart watch 14 positioned on the back hand side 58 of the wrist 18. The user 20 can engage with the glucose reader 38 by wirelessly connecting the glucose reader 38 with the smart watch 14 by an application. The glucose reader 38 uses the pair of infrared diodes 50 to measure the blood glucose levels without puncturing the skin to obtain blood from the user 20. The circuit board 54 sends the data to the smart watch 14 wherein the user 20 can view the blood glucose levels within the wrist 18.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A glucose monitoring watch device configured to obtain glucose levels from the wrist of the user, the glucose monitoring watch device comprising:
a wristband comprising a smart watch, said wristband being configured for enwrapping the wrist of the user, a first side of said wristband having a plurality of holes, a second side of said wristband having an aperture, each of said holes being a first portion of a wristband fastener;
a glucose reader being a second portion of said wristband fastener, said glucose reader further including:
an encasing having a top end and a bottom end, said encasing being cylindrical and having an interior, said interior defining a space for a pair of infrared diodes to be positioned within, each of said infrared diodes being configured to measure glucose levels within the wrist of the user;
a pair of glass caps being positioned on said top end of said encasing, said pair of glass caps being configured for emitting infrared light upon the wrist of the user; and
a circuit board being positioned on said bottom end of said encasing, said circuit board being in electric communication with said pair of infrared diodes, said circuit board being configured for sending readings from said pair of infrared diodes to said smart watch.

2. The glucose monitoring watch device of claim 1, further comprising said smart watch being configured for being a mobile device with touch screen capabilities.

3. The glucose monitoring watch device of claim 1, further comprising said smart watch having a pair of sides.

4. The glucose monitoring watch device of claim 3, further comprising said wristband protruding out from each of said sides of said smart watch.

5. The glucose monitoring watch device of claim 1, further comprising said wristband having a wrist surface and a watch surface.

6. The glucose monitoring watch device of claim 5, further comprising said wrist surface of said wristband being configured for abutting a surface of the wrist of the user.

7. The glucose monitoring watch device of claim 6, further comprising said watch surface of said second side of said wristband being positioned abutting said wrist surface of said first side of said wristband.

8. The glucose monitoring watch device of claim 7, further comprising said glucose reader being configured for being positioned within said aperture of said second side of said wristband and an in-use hole of said plurality of holes of said first side of said wristband wherein retaining the wristband in a fixed positioned around the wrist of the user.

9. The glucose monitoring watch device of claim 1, further comprising each of said glass caps being coupled to a respective one of each of said infrared diodes.

10. The glucose monitoring watch device of claim 1, further comprising said circuit board being a transceiver, said circuit board being in wireless communication to said smart watch.

11. A glucose monitoring watch device configured to obtain glucose levels from the wrist of the user, the glucose monitoring watch device comprising:
a wristband comprising a smart watch, said smart watch being configured for being a mobile device with touch screen capabilities, said smart watch having a pair of sides, said wristband protruding out from each of said sides of said smart watch, said wristband being configured for enwrapping the wrist of the user, said wristband having a wrist surface and a watch surface, said wrist surface of said wristband being configured for abutting a surface of the wrist of the user, a first side of said wristband having a plurality of holes, a second side of said wristband having a aperture, each of said holes being a first portion of a wristband fastener, said watch surface of said second side of said wristband being positioned abutting said wrist surface of said first side of said wristband;
a glucose reader being a second portion of said wristband fastener, said glucose reader being configured for being positioned within said aperture of said second side of said wristband and an in-use hole of said plurality of holes of said first side of said wristband wherein retaining the wristband in a fixed positioned around the wrist of the user, said glucose reader further including:
an encasing having a top end and a bottom end, said encasing being cylindrical and having an interior, said interior defining a space for a pair of infrared diodes to be positioned within, each of said infrared diodes being configured to measure glucose levels within the wrist of the user;
a pair of glass caps being positioned on said top end of said encasing, each of said glass caps being coupled to a respective one of each of said infrared diodes, said pair of glass caps being configured for emitting infrared light upon the wrist of the user; and
a circuit board being positioned on said bottom end of said encasing, said circuit board being a transceiver, said circuit board being in electric communication with said pair of infrared diodes, said circuit board being in wireless communication to said smart watch, said circuit board being configured for sending readings from said pair of infrared diodes to said smart watch.
